(19) United States
(12) Patent Application Publication
(10) Pub. No.: US 2008/0057110 A1
(43) Pub. Date: Mar. 6, 2008

(54) Compositions for alcoholic beverages and methods of producing thereof

(76) Inventor: Alexander Skirpa, Rockville, MD (US)

Correspondence Address: SHAILAJA SHIRODKAR, 2 CHESTER MILL COURT, SILVER SPRING, MD, 20906, US

(21) Appl. No.: 11/895,816

(22) Filed: Aug. 28, 2007

Related U.S. Application Data
(60) Provisional application No. 60/840,769, filed on Aug. 29, 2006.

(51) Int. Cl. A23L 1/30; A23L 1/302; A61K 47/00; C12C 11/11

(52) U.S. Cl. 424/439; 426/11; 426/72; 426/74; 426/89

(57) ABSTRACT

Compositions and methods of producing an alcoholic beverage are provided that reduce and or prevent the after-effects of alcohol consumption and protect human organs during and after consumption of alcohol.

### CLAIM OF PRIORITY

This application claims the benefit of U.S. Application No. 60/840,769 filed on Aug. 29, 2006, which is incorporated by reference in its entirety.

### FIELD OF INVENTION

The present invention relates to compositions for alcoholic beverages and method of producing the same. More specifically, the invention relates to alcoholic beverages, and methods of producing the same which can prevent the after-effects of alcohol consumption and methods of producing alcoholic beverages, which can prevent organ damage and other alcohol related health problems in addition to replenishing depleted nutrients (vitamins and minerals).

### BACKGROUND OF INVENTION

U.S. Patent Application No. 20030157218 by E. T. Donhowe relates to preparation of a sport beer or malt beverage that enhances nutrition in comparison to existing beer or malt beverages. The patent application does not describe compositions or methods thereof for preventing the after-effects of alcohol consumption or methods of preventing organ damage.

Vitamin-fortified beer has been described at the website Realbeer.com (http://www.realbeer.com/news/articles/news-001675.php) wherein thiamin was added to beer because it might reduce alcohol-related problems.

### SUMMARY OF INVENTION

Compositions and methods of producing an alcoholic beverage are provided that can reduce the after-effects of alcohol consumption, alleviate the corrosive effects while consuming the alcohol and protect human organs during and after consumption thereof.

In one aspect of the invention, an alcoholic beverage comprises:

(a) an alcoholic drink;

(b) from about 1 mg to about 7000 mg antioxidants;

(c) from about 1 mcg to about 1000 mg minerals;

(d) from about 1 mg to about 3000 mg amino acids; and

(e) an encapsulate, the encapsulate in an amount sufficient to encapsulate at least a substantial portion of the antioxidants, minerals and amino acids.

Yet another aspect of the invention includes the method for suppressing and or eliminating the after-effects of alcohol consumption comprises:

contacting an alcoholic drink with a composition comprising:

(a) from about 1 mg to about 7000 mg antioxidants;

(b) from about 1 mcg to about 1000 mg minerals;

(c) from about 1 mg to about 3000 mg amino acids; and

(d) an encapsulate, the encapsulate in an amount sufficient to encapsulate at least a substantial portion of the antioxidants, minerals and amino acids to make an alcoholic beverage.

Another aspect of the invention includes a method of protecting human organs during consumption of alcohol comprising: contacting an alcoholic drink with compositions described herein.

Another aspect of the invention includes a composition useful for preventing and treating the symptoms of alcohol hangover comprises:

(a) at least one antioxidant component;

(b) at least one mineral component;

(c) at least one amino acid component; and

(d) an encapsulate component, the encapsulate in an amount sufficient to encapsulate at least a substantial portion of the antioxidants, minerals and amino acids whereby said components are added to an alcoholic drink.

Some advantages of the invention include encapsulating the nutrients such that taste of the alcoholic beverage is not affected or altered appreciably in any way. Upon ingestion, the body can have immediate access to essential nutrients, which are being continuously depleted during the consumption of alcohol. Therefore, the deleterious effects of consumption of alcohol can be ameliorated while providing rapid relief to the affected individual.

Other features and advantages of the invention will be apparent from the following detailed description.

### DESCRIPTION OF DRAWINGS

FIG. 1 is a graph illustrating the benefits of consuming alcohol with the alcoholic beverages including compositions of the invention.

### DEFINITION OF TERMS

An alcoholic drink is any commercially available liquid containing alcohol including beers, lager, cider, wine, carbonated bottled mixed drinks and non-carbonated mixed drinks, among others.

An alcoholic beverage is the alcoholic drink and the compositions of the invention.

### DETAILED DESCRIPTION OF THE INVENTION

People who consume alcohol drinks to intoxication often experience unpleasant physical and mental symptoms. Physical symptoms include fatigue, headache, increased sensitivity to light, redness of the eyes, muscle aches, and dry mouth. Some sympathetic nervous system symptoms can include an increased systolic blood pressure, rapid heartbeat, tremors and sweating. Dizziness, vertigo (a sense of the room spinning), and mood disturbances such as depression, irritability and anxiety can be signs of mental symptoms affecting the brain. These after-effects of alcohol consumption are collectively called a hangover.

The particular symptoms of a hangover can vary in their intensity based on the person's physical condition as well as the amount and type of alcohol consumed. Typically, a hangover begins within several hours after drinking alcoholic drinks has ceased and may continue for several hours thereafter. Within the body, alcohol causes the blood vessels to swell and dilate, increasing the pressure on the brain. The diuretic property of alcohol causes dehydration by removing water out of the body. These symptoms are caused by the production of toxic acetaldehyde produced by the metabolism of alcohol by alcohol dehydrogenase and partly due to dehydration. The brain shrinks away from the skull due to dehydration triggering pain sensors in the outer surface of the brain leading to a headache. Other components in a drink such as tannins in red wine can also contribute to a hangover.

However, despite the prevalence of hangovers, this condition is not well understood. Typically, at onset a person's blood concentration level (BAC) is about zero when the symptoms of a hangover can emerge. Besides feeling ill, a hangover can impair a person's ability to perform a task thus increasing the risk of injury. Besides suffering from dehydration and toxic by-products of alcohol metabolism, the body can suffer a loss of essential nutrients. Although time is the best remedy for a hangover, a good hangover remedy can ameliorate the after-effects of alcohol consumption by replacing essential nutrients lost by the body while and during being under the influence of alcohol, as described in the present invention. The after-effects of alcohol, during and after consumption can be neurological, cellular, or functional among others and can include liver, skin and eye damage. The present invention includes compositions for alcoholic beverages and methods of producing thereof.

The present invention provides for alcoholic beverage compositions including:

(a) an alcoholic drink;

(b) from about 1 mg to about 7000 mg antioxidants;

(c) from about 1 mcg to about 1000 mg minerals;

(d) from about 1 mg to about 3000 mg amino acids; and

(e) an encapsulate, the encapsulate in an amount sufficient to encapsulate at least a substantial portion of the antioxidants, minerals and amino acids.

Alcoholic drinks in the commercial market place include beers, lager, cider, wine, carbonated bottled mixed drinks and non-carbonated mixed drinks, among others. The alcoholic content in alcoholic drinks can be measured by alcoholic proof which is a measure of how much ethanol is in an alcoholic drink, and is approximately twice the percentage of alcohol by volume (ABV, the unit that is commonly used presently). The proof number is twice the percentage of the alcohol content measured by volume at a temperature of 60° F. (15.5° C.). Therefore “80 degrees proof” is 40% alcohol by volume (most of the other 60% is water). If a 150-proof drink is mixed half-and-half with water, the product is 75 proof. The alcoholic drink can be 5 to 200 degrees proof. The present invention contemplates contacting the alcoholic drinks with the compositions disclosed herein.

Antioxidants protect cells from the damage caused by unstable molecules known as free radicals. Antioxidants donate an electron to the free radical and convert it to a harmless molecule, thus antioxidants intercept free radicals and protect cells from the oxidative damage that leads to disease and prevent some of the damage from free radicals after and during consumption. The antioxidants can include vitamin A, vitamin C, vitamin E, vitamin B (for example, vitamins B₁, B₂, B₃, B₅, B₆, B₇, B₉ and B₁₂), carotenoids, and other substances. Suitable examples of vitamins as antioxidants include Vitamin C, Vitamin E, and Vitamin B₁. Peptides, for example, tripeptides can also act as antioxidants such as glutathione (g-glutamylcysteinylglycine, GSH) which is a sulfhydryl (—SH) antioxidant, antitoxin, and enzyme cofactor. Glutathione exists in two forms, the antioxidant “reduced glutathione” tripeptide is conventionally called glutathione and abbreviated GSH; the oxidized form is a sulfur-sulfur linked compound, known as glutathione disulfide or GSSG Suitable example of a peptide antioxidant includes glutathione, reduced or oxidized. Note that 1 IU Vitamin E is the biological equivalent of about 0.667 mg d-alpha-tocopherol (⅔ mg exactly), or of 1 mg of dl-alpha-tocopherol acetate. Therefore, 100 IU of vitamin E in the composition is approximately 66.7 mg of d-alpha tocopherol or 100 mg of dl-alpha-tocopherol. The antioxidants can be in amount from about 1 mg to about 7000 mg.

Dietary minerals are the chemical elements required by living organisms, other than the four elements Carbon, Hydrogen, Nitrogen, and Oxygen which are ubiquitous in organic molecules. They can be either bulk minerals (required in relatively large amounts) or trace minerals (required only in very small amounts). Bulk minerals can include calcium and potassium. Trace minerals can include chromium, cobalt, copper, fluorine, iodine, iron, manganese, molybdenum, selenium and zinc. Suitable examples of minerals can include selenium, potassium and calcium. The minerals can be in the amount from about 1 mcg to about 1000 mg.

An amino acid is any molecule that contains both amino and carboxylic acid functional groups. Twenty amino acids are encoded by the standard genetic code and are called standard amino acids. Among the twenty amino acids are arginine, glutamic acid, glycine, cysteine, N-acetyl-L-cysteine and glutamic acid. Suitable examples of amino acids include cysteine and N-acetyl-L-cysteine. The amino acids can be in the amount from about 1 mg to about 3000 mg.

An encapsulate can be any medium which can be safely consumed by humans. The encapsulate encompasses or embeds the compositions disclosed herein. For example, the encapsulate can be a gel, or a coating or any other method of encapsulation. Gel entrapment is one of the most used encapsulation techniques because of its mildness, ease of operation and applicability. A hydrocolloid capsule is an example of liquid/nutrient encapsulated in a spherical or non spherical polymer membrane. The encapsulate can be a hydrocolloid. The encapsulation can be by, for example, gel entrapment, polymer films or microencapsulation.

Microencapsulation is a process in which tiny pieces of an ingredient are packaged, or encapsulated, within another material in order to protect the active ingredient from the surrounding environment. Encapsulated active ingredients provide taste masking and protection from other surrounding materials and can include oils, proteins, hydrocolloids, gums, waxes and polymer resins. Encapsulation methods can include physical methods, for example, air suspension coating, stationary extrusion, centrifugal extrusion, nozzle extrusion, vibrating nozzle, rotating disk, pan coating, fluid bed and spray drying. Encapsulation by chemical methods can include, for example, simple or complex coacervation, phase separation, interfacial polymerization, matrix polymerization, interfacial polymerization, solvent evaporation, in situ polymerization, liposome, sol-gel methods and nanoencapsulation. Capsules can range in size from <1 micron to several millimeters. The encapsulate can be a digestible oil, for example, a vegetable oil.

Even when the aim of a microencapsulation application is the isolation of the core from its surrounding, the wall must be ruptured at the time of use. Many walls are ruptured easily by pressure or shear stress, as in the case of breaking dye particles during writing to form a copy. Capsule contents may be released by melting the wall, or dissolving it under particular conditions, as in the case of an enteric drug coating. In other systems, the wall is broken by solvent action, enzyme attack, chemical reaction, hydrolysis, or slow disintegration. An enteric coating is a barrier applied to the composition that controls the location in the digestive system where it is absorbed.

In another embodiment, the present invention provides a method for suppressing the after-effects of alcohol consumption comprising:

contacting an alcoholic drink with a composition comprising:

(a) from about 1 mg to about 7000 mg antioxidants;

(b) from about 1 mcg to about 1000 mg minerals;

(c) from about 1 mg to about 3000 mg amino acids; and

(d) an encapsulate, the encapsulate in an amount sufficient to encapsulate at least a substantial portion of the antioxidants, minerals and amino acids to make an alcoholic beverage.

Upon ingestion, the alcohol affects many organs in the body such as, for example, skin, eyes and perhaps most notably the liver. The liver contains an enzyme, alcohol dehydrogenase, which breaks down the alcohol into acetaldehyde, which in turn is broken down into acetic acid. The acetic acid is converted to fats or carbon dioxide and water. Chronic drinkers tax the liver heavily which leads to liver cirrhosis. Liver cirrhosis resulting from alcohol abuse is one of the leading causes of death in the United States. There is a critical need for any type of alcoholic beverage which protects the liver of alcoholics, college students and drinkers alike during consumption of alcohol. The present invention provides such compositions for alcoholic beverages with and without encapsulation. The present invention also provides a method for protecting human organs during consumption of alcoholic drinks using the compositions described herein.

The compositions of the invention can be delivered to the alcoholic beverage just prior to consumption to make the composition available within the alcoholic drink. This delivery system could be similar to the Guinness® floating widget found in cans of Guinness beer (http://en.wikipedia.org/wiki/Widget₁₃%28beer %29) for a more detailed description. Similarly, compositions can be included in a pressurized hollow container or sphere that releases the compositions into the alcoholic beverage after the beverage container is opened.

The present invention contemplates a method for protecting human organs during consumption of alcohol comprising of contacting the alcoholic drink with the compositions described above. Liver cirrhosis resulting from alcohol abuse is one of the leading causes of death in the United States. There is a critical need for any type of alcoholic beverage which can protect the liver of alcoholics, college students and drinkers alike during consumption of alcohol.

The present invention includes compositions useful for preventing and treating the symptoms of alcohol hangover comprising:

(a) at least one antioxidant component;

(b) at least one mineral component;

(c) at least one amino acid component; and

(d) an encapsulate component, the encapsulate in an amount sufficient to encapsulate at least a substantial portion of the antioxidants, minerals and amino acids whereby said components are added to an alcoholic drink.

Examples of compositions and methods of testing the invention for reducing the symptoms of alcohol related illnesses and hangovers are given to illustrate the composition and are not limited to the examples shown.

### EXAMPLE 1

| Composition | Weight | % Daily Value |
|---|---|---|
| Calcium | 100 mg | 10% |
| Cysteine | 1000 mg | * |
| Glutathione | 50 mg | * |
| Potassium | 33 mg | 1% |
| Selenium | 27 mcg | 39% |
| Vitamin B₁ | 1100 mg | 73333% |
| Vitamin C | 3500 mg | 5833% |
| Vitamin E | 100 IU | 333% |

The quantities listed in the above composition can be a microencapsulated target dose. If the composition is to be used in a 6-pack of beer, then one can divide the target dose by 6 and that would be the amount that is listed on each of the individual beer labels. Alternatively, the target dose can be placed in one can/bottle of beer for consumption. Alternatively, a single shot of vodka with 100% of the target dose could be served in bars as a protective shot/or chaser. The target dose can be contained in a small container.

### EXAMPLE 2

### Method of Testing

Seven subjects participated voluntarily in the testing of beer with and without the compositions. The tests were done on two separate occasions at least three (3) days apart. In the first session, a placebo alcoholic drink was served and in the second session the alcoholic beverage with the composition in the alcoholic drink was served. The drink chosen for the test was 12 ounce can of Budweiser beer. Each subject consumed enough 12 ounce cans of beer, at their discretion, to cause each a hangover. The subjects ingested the placebo and alcoholic beverage on an empty stomach at least three hours after a meal. Each subject drank enough beer to cause a hangover in that particular subject.

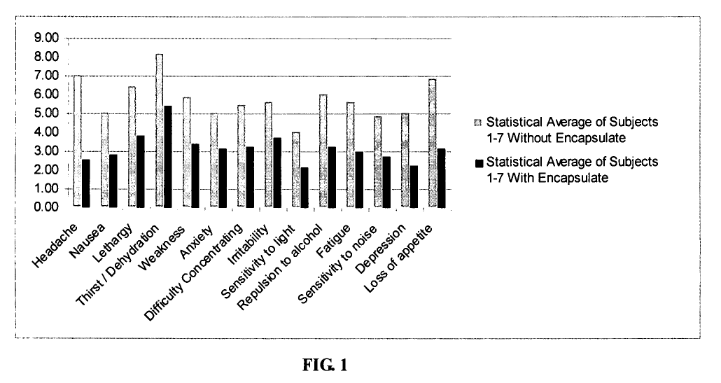

Referring to FIG. 1, 12-14 hours after each occasion the subjects filled out the questionnaire to reflect each subject's observation of hangover symptoms. Each subject was asked to rate the hangover symptoms after consumption of the alcoholic drink with and without the composition. Hangover symptoms that were rated included, for example, headache, nausea, lethargy, thirst/dehydration, weakness, anxiety, difficulty concentrating, irritability, sensitivity to light, repulsion to alcohol, fatigue, sensitivity to noise, depression and loss of appetite on a scale of 1 to 10 as shown in Table 1.

TABLE 1

| | Statistical Average | Statistical Average |
|---|---|---|
| | of Subjects 1-7 | of Subjects 1-7 |
| Symptom of Hangover | Without Encapsulate | With Encapsulate |
| Headache | 7.00 | 2.57 |
| Nausea | 5.00 | 2.86 |
| Lethargy | 6.43 | 3.86 |
| Thirst/Dehydration | 8.14 | 5.43 |
| Weakness | 5.86 | 3.43 |
| Anxiety | 5.00 | 3.14 |
| Difficulty Concentrating | 5.43 | 3.29 |
| Irritability | 5.57 | 3.71 |
| Sensitivity to light | 4.00 | 2.14 |
| Repulsion to the thought of | 6.00 | 3.29 |
| alcohol | | |
| Fatigue | 5.57 | 3.00 |
| Sensitivity to noise | 4.86 | 2.71 |
| Depression | 5.00 | 2.29 |
| Loss of appetite | 6.86 | 3.14 |

Taste tests were also conducted on each subject as shown in Table 2. Each subject was asked whether the sample tasted like beer, tasted extra sweet, tasted extra sour and finally tasted extra bitter. The results in Table 2 indicate that on average the seven subjects gave the alcoholic beverage that included the composition on average a rating of 7.57 on a scale of 10, with 10 tasting exactly like beer.

TABLE 2

| | Average for |
|---|---|
| TASTE TEST RESULTS | all subjects |
| Tastes like beer | 7.57 |
| Aside from the taste of beer, I noticed some extra sweet | 0.00 |
| taste | |
| Aside from the taste of beer, I noticed some extra salty taste | 1.57 |
| Aside from the taste of beer, I noticed some extra sour taste | 1.00 |
| Aside from the taste of beer, I noticed some extra bitter | 0.86 |
| taste | |

As can be seen from the data shown in Table 1 and FIG. 1 consumption of the alcoholic beverage with the composition is able to alleviate the after effects of a hangover and significantly reduce hangover symptoms. Moreover, the taste test results shown in Table 2 indicate that the addition of the composition to the alcoholic drink does not significantly alter the taste of the alcoholic drink.

What is claimed:

1. An alcoholic beverage comprising:
(a) an alcoholic drink;
(b) from about 1 mg to about 7000 mg antioxidants;
(c) from about 1 mcg to about 1000 mg minerals;
(d) from about 1 mg to about 3000 mg amino acids; and
(e) an encapsulate, the encapsulate in an amount sufficient to encapsulate at least a substantial portion of the antioxidants, minerals and amino acids.

2. The alcoholic beverage according to claim 1, wherein the alcoholic drink is selected from the group consisting of beer, lager, wine, cider, carbonated mixed drink and uncarbonated mixed drink.

3. The alcoholic beverage according to claim 1, wherein the alcohol content is in the range 5 to about 200 degrees proof.

4. The alcoholic beverage according to claim 1, wherein the antioxidants are selected from the group consisting of vitamin C, vitamin E, vitamin B₁ and glutathione.

5. The alcoholic beverage according to claim 1, wherein the mineral is selected from the group consisting of chromium, cobalt, copper, fluorine, iodine, iron, manganese, molybdenum, selenium, calcium, potassium and zinc.

6. The alcoholic beverage according to claim 5, wherein the minerals are selenium, calcium and potassium.

7. The alcoholic beverage according to claim 1, wherein the amino acid is cysteine.

8. The alcoholic beverage according to claim 1, wherein the encapsulate is a vegetable oil.

9. The alcoholic beverage according to claim 1, wherein the encapsulate is an enteric coating.

10. The alcoholic beverage according to claim 1, wherein the encapsulate is prepared by microencapsulation.

11. A method for suppressing the after-effects of alcohol consumption comprising:
contacting an alcoholic drink with a composition comprising:
(a) from about 1 mg to about 7000 mg antioxidants;
(b) from about 1 mcg to about 1000 mg minerals;
(c) from about 1 mg to about 3000 mg amino acids; and
(d) an encapsulate, the encapsulate in an amount sufficient to encapsulate at least a substantial portion of the antioxidants, minerals and amino acids to make an alcoholic beverage.

12. The method according to claim 11 wherein the alcoholic beverage retains the taste of the alcoholic drink.

13. A method of protecting human organs during consumption of alcohol comprising: contacting an alcoholic drink with compositions of claim 1.

14. A composition useful for preventing and treating the symptoms of alcohol hangover comprising:
(a) at least one antioxidant component;
(b) at least one mineral component;
(c) at least one amino acid component; and
(d) an encapsulate component, the encapsulate in an amount sufficient to encapsulate at least a substantial portion of the antioxidants, minerals and amino acids whereby said components are added to an alcoholic drink.

15. The composition according to claim 14, wherein the antioxidants are selected from the group consisting of vitamin C, vitamin E, vitamin B₁ and glutathione.

16. The composition according to claim 14, wherein the mineral is selected from the group consisting of chromium, cobalt, copper, fluorine, iodine, iron, manganese, molybdenum, selenium, calcium, potassium and zinc.

17. The composition according to claim 16, wherein the minerals are selenium, calcium and potassium.

18. The composition according to claim 14, wherein the amino acid is cysteine.

19. The composition according to claim 14, wherein the encapsulate is a vegetable oil.
